(19) United States
(12) Patent Application Publication
(10) Pub. No.: US 2008/0318825 A1
(43) Pub. Date: Dec. 25, 2008

(54) Hair Treatment Agent

(75) Inventor: Jan Baumeister, Farvagny-le-Petit (CH)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Proctor & Gamble Company, Cincinnati, OH (US)

(21) Appl. No.: 11/885,717

(22) PCT Filed: Nov. 23, 2005
(86) PCT No.: PCT/EP05/12545

§ 371 Date: Jun. 10, 2008

(30) Foreign Application Priority Data
Nov. 24, 2004 (DE) 10 2004 056 758.1

(51) Int. Cl. A61K 8/58

(52) U.S. Cl. 510/122

(57) ABSTRACT

The subject matter of the present invention is a hair treatment agent that can be used for example as a hair cleaning agent or as a hair rinse, and is preferably present in the form of an optically clear product. The hair treatment agent according to the present invention contains hydrophilic silicones and hydrophobic silicones.

The subject matter of the present invention is a composition that can be used for example as a hair cleaning agent and is preferably provided in the form of an optically clear product. The hair treatment agent according to the present invention contains hydrophilic silicones and hydrophobic silicones, and may also contain cationically active hair conditioning materials.

Cosmetic preparations, above all those used for cleaning hair and the body, such as shower gels, foam baths, shampoos, and other hair care products, are based mainly on anionic surfactants such as alkyl sulfates, alpha-olefin sulfonates, and alkylether sulfates. The primary demand placed on such agents is the removal of sweat, grease, and dirt particles on the skin and in the hair. When washing hair, a problematic effect has often been that the combability of the hair is drastically worsened, and in addition the optical, and often also haptic, appearance or condition of the hair is no longer acceptable.

As cosmetic rinsing products, in the 1980s what are called two-in-one shampoos were introduced, and succeeded in winning significant market share in the early 1990s. These shampoos, which contain emulsified silicone oils, had the effect of conditioning the hair as well as cleaning it. However, silicone oil emulsions with hydrophobic silicone compounds have exhibited problems with regard to compatibility and stability; they have a strong lather-reducing effect, and in addition they are in general not transparent.

For these reasons, hydrophilic silicone polyethers were introduced into the market. But, apart from their generally higher price, the conditioning effect of hydrophilic silicone polyethers on the skin and hair has generally been considered to be much less than the conditioning effect of hydrophobic silicone oils.

With these problems in mind, attempts were carried out to provide aqueous compositions containing hydrophobic silicone oils, in which the silicone oil was present in a solubilized or microemulsified state.

EP B1 0 529 883 discloses shampoo compositions containing sodium lauryl ether sulfate and cocoamide propylbetaine as surfactants and 1.0 wt % silicone oil. The silicone oil is added as a microemulsion created using an emulsion polymerisation technique.

DE 100 53 728 A1 describes an optically transparent aqueous composition comprising a hydrophobic silicone oil in a quantity from 1 to 3 wt % in relation to the total weight of the composition, a solubilizer for the silicone oil, and an anionic surfactant.

DE 100 53 727 A1 describes an optically transparent aqueous composition comprising a hydrophobic silicone oil and an alkylethercarboxylate.

U.S. Pat. No. 4,933,176 relates to optically clear shampoo compositions that contain anionic surfactants, nonionic surfactants, a saccharine salt, water, and a silicone component having polydiorganosiloxanes or cyclodiorganosiloxanes.

EP A1 1 356 803 discloses a hair treatment composition comprising a blend of organomodified silicones which deposits on hair. This is achieved by operating with organomodified silicones within a defined hydrophilicity range.

EP A1 1 356 802 describes a fiber treatment composition which deposits on a variety of fibers, especially hair, of different levels of damage. This is achieved by operating with organomodified silicones within a defined hydrophilicity range.

EP A1 1 081 272 refers to siloxanes having amine, polyol and amide-functionalities and to a fiber treatment composition containing them. The composition is preferably formulated as an aqueous emulsion.

WO 02/22084 A2 discloses a concentrated hair conditioning composition for preparing a treated water having a silicone conditioning agent concentration of 0.001% to 2% for applying to the hair, comprising from about 0.01% to about 20% by weight of a silicone conditioning agent, and an aqueous carrier, wherein the composition is capable of providing a deposition of from about 10 ppm to about 5000 ppm of silicone conditioning agent when applied to the hair as the treated water.

A disadvantage of the above-named compositions known from the prior art is the fact that the properties of the hair leave something to be desired in some instances after an application of the described compositions. For example, in some instances the dry hold was rated as unsatisfactory. In addition, hair treated using the agents known from the prior art often show, for example, unsatisfactory shine.

In addition, with the use of hydrophobic silicones, in particular their use in shampoos, there are often problems with the lathering ability of the formulas. Up to now, in order to avoid this disadvantage the content of active washing and foaming substances was drastically increased. Thus, for example DE 100 53 727 A1 and DE 100 53 728 A1 each describe a content of at least 10 wt % active washing substance; preferably, however, in order to achieve a sufficient foaming capacity approximately 25 wt % active washing substances are used here. However, the tolerability of the shampoos often suffers as a result, and they are often perceived as “hard” by the user. Such shampoos are often also considered by the user to lack a conditioning effect.

The object thus arose of providing a composition suitable for use as a hair treatment agent that fulfills at least the typical demands made on a hair treatment agent with respect to hair cleaning and/or hair conditioning, and is simultaneously provided in an optically appealing form.

The above-named objects, and additional objects resulting for those skilled in the art from the disclosure of the present application, are achieved by a composition containing a combination of hydrophilic and hydrophobic silicone compounds.

The subject matter of the present invention is therefore a composition containing

a) 0.01 to 10 wt % of a hydrophobic silicone compound, and

b) 0.01 to 15 wt % of at least one hydrophilic silicone compound, and

c) water, as well as at least one compound selected from the group made up of anionic surfactants, nonionic surfactants, cationic surfactants, polymers having cationic or cationisable groups, cationically derived proteins, cationically derived protein hydrolysates, and betaines.

A hydrophobic silicone oil is in general a silicone oil that is soluble in paraffin oil at 25° C. (77° F.). In the context of the present invention, a silicone oil is designated as hydrophilic if at least approx. 0.1 g/L (0.003 g/oz) water, for example at least approx. 0.2, 0.3, 0.4, 0.5, 0.6, 0.7, 0.8, 0.9, 1, 2, 3, 5, 10, or at least approx. 20 g/L (0.6 g/oz) water dissolve in water at 25° C. (77° F.).

Hydrophobic silicone oils used according to the present invention include both volatile and also nonvolatile silicone oils. Suitable oils are for example cyclic methylsiloxanes having the formula ((R)₂SiO)ₓ, where R stands for a linear or branched, saturated or unsaturated alkyl group having 1 to 6 C atoms or a mono- or polycyclic cycloalkyl or heterocycloalkyl group having 4 to 8 C atoms or an aromatic or araliphatic group having 6 to 10 C atoms, and x stands for a number from 3 to 6, or straight chain methylsiloxanes having the formula ((R)₂SiO{(R)₂SiO}_{y}Si(R)₃, the groups R having, independent of one another, the meaning already cited above, and y standing for a number from 0 to 5.

Suitable cyclic methylsiloxanes are for example hexamethylcyclotrisiloxane, a solid material having a boiling point of 134° C. (273° F.) and the formula {Me₂SiO}₃, where Me stands for a methyl group; octamethylcyclotetrasiloxane, with a boiling point of 176° C. (349° F.), a viscosity of 2.3 mm²/s and the formula {(Me₂)SiO}₄; decamethylcyclopentasiloxane (cyclomethicone), having a boiling point of 210° C. (410° F.), a viscosity of 3.87 mm²/s and the formula {(Me₂)SiO}₅; and dodecamethylcyclohexasiloxane, having a boiling point of 245° C. (473° F.), a viscosity of 6.62 mm²/s and the formula {(Me₂)SiO}₆. Suitable straight chain methylsiloxanes are for example hexamethyldisiloxane, with a boiling point of 100° C. (212° F.), a viscosity of 0.65 mm/s and the formula Me₃SiOSiMe₃; octamethyltrisiloxane, with a boiling point of 152° C. (306° F.), a viscosity of 1.04 mm/s and the formula Me₃SiOMe₂SiOSiMe₃; decamethyltetrasiloxane, having a boiling point of 194° C. (381° F.), a viscosity of 1.53 mm²/s and the formula Me₃SiO(Me₂SiO)₂SiMe₃; dodecamethylpentasiloxane, having a boiling point of 229° C. (444° F.), a viscosity of 2.06 mm²/s and the formula Me₃SiO(Me₂SiO)₃SiMe₃; tetradecamethylhexasiloxane, having a boiling point of 245° C. (473° F.), a viscosity of 2.63 mm²/s and the formula Me₃SiO(Me₂SiO)₄SiMe₃; and hexadecamethylheptasiloxane, having a boiling point of 270° C. (518° F.), a viscosity of 3.24 mm²/s and the formula Me₃SiO(Me₂SiO)₅SiMe₃.

In addition, long chain and straight chain siloxanes, such as for example phenyltrimethicone, bis(phenylpropyl)dimethicone, dimethicone, and dimethiconol are included.

The above-named silicone compounds can in principle have essentially arbitrary substituents, as long as these substituents do not unavoidably counteract the intended purpose of use.

Also suitable for use in the context of the present invention as hydrophobic silicones are trialkylsiloxysilicates and trialkylsiloxypolysilicates. Such compounds follow the general formula R¹ₙSiO₍₄₋ₙ₎₂ with 1<n<1.5, wherein the groups R¹ stand, independently of one another, for a linear or branched, saturated or unsaturated alkyl group having 1 to 6 C atoms, or a mono-or polycyclic cycloalkyl- or heterocycloalkyl group having 4 to 8 C atoms, or an aromatic or araliphatic group having 6 to 10 C atoms.

Functionalized silicones that can be contained in compositions according to the present invention can for example contain organomodified silicones of the graft type; polar functional substituents can be contained in or on monovalent organic groups, hereinafter referred to as A¹, A², A³ and A⁴, as follows:

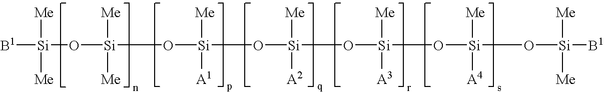

Likewise, for example organomodified silicones of the block copolymer type can be contained; polar functional substituents in or on monovalent organic groups, hereinafter referred to as A¹, A², A³ and A⁴, are suitable.

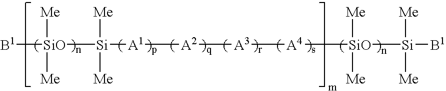

Here, Me stands for methyl, m is greater than or equal to 1, n is approximately 50 to 2,000, p is approximately 0 to 50, q is approximately 0 to 50, r is approximately 0 to 50, s is approximately 0 to 50, p+q+r+s being greater than or equal to 1, B¹ stands for H, OH, an alkyl or alkoxy group.

The above functionalized silicones of the graft or block copolymer type can also contain silicone branched groups, including MeSiO_{3/2}, known as silsesquioxane or T-groups, and SiO_{4/2}, known to those skilled in the art as Q-groups.

The organic groups A¹, A², A³ and A⁴ can be straight chain, branched, or mono- or polycyclic aliphatic, simple or complex unsaturated alkyl, aryl, heteroalkyl, heteroaliphatic or heteroolefin groups comprising 3 to 150 carbon atoms together with 0 to 50 heteroatoms, in particular O, N, S, P, and can contain one or more polar substituents, to be selected from electron-accepting, electron-neutral, or electron-supplying groups with Hammett σ(para) values between −1 and +1.5, which can be nonionic, zwitterionic, cationic or anionic, comprising for example groups α₁, α₂, α₃, and α₄, as defined below; S-bound groups containing Sα¹, SCN, SO₂ α¹, SO₃α¹, SSα1¹, SOα¹, SO₂Nα¹α², SNα¹α², S(Nα¹) α², S(O)(Nα¹) α², Sα¹(Nα²), SONα¹α²; O-bound groups containing Oα¹, OOα¹, OCN, ONα¹α²; N-bound groups containing Nα¹α², Nα¹α²α³+, NC, Nα¹Oα², Nα¹Sa², NCO, NCS, NO₂, N=Nα¹, N=NOα¹, Nα¹CN, N=C=Nα¹, Nα¹Nα²α³, Nα¹Nα²Nα³α⁴, Nα¹N=Nα²; various other groups containing COX, CON₃, CONα¹α², CONα¹COα², C(=Nα¹)Nα¹α², CHO, CHS, CN, NC, and X.

In the above formulas, α₂, α₂, α₃, and α₄ can stand for straight chain, branched, or mono- or polycyclic aliphatic, simple or complex unsaturated alkyl, aryl, heteroalkyl, heteroaliphatic or heteroolefin groups comprising 3 to 150 carbon atoms together with 0 to 50 heteroatoms, in particular O, N, S, P. X stands for F, Cl, Br, or I. Hammett-σ(para)-values are discussed under the entry “Hammett Gleichung [Hammett Equation],” in: R\ddot{m}pp Chemie Lexikon, Georg Thieme Verlag, Stuttgart, New York, 9^{th} Edition, 1995.

Preferred polar functional substituents for use in the present invention, as described, contain (but are not limited to) polyoxyalkylenes (polyethers), primary and secondary amines, amides, quaternary ammonium, carboxyl, sulfonate, sulfate, carbohydrate, phosphate, and hydroxyl. More preferably, the polar functional substituents of the present invention contain (but are not limited to) polyoxyalkylenes, primary and secondary amines, amides and carboxyl.

Suitable functionalized silicones corresponding to the present invention contain (but are not limited to) organomodified silicones having amine functionality, which are commercially available under trade names such as ADM1100 and ADM1600 of Wacker Silicones, DC2-8211, DC8822, DC8822A, DC8803, DC2-8040, DC2-8813, DC2-8630 and DC8566 of Dow Corning Corporation, KF-862, KF-861, KF-862S, KF-8005, KF-8004, KF-867S, KF-873, and X-52-2328 of Shin-Etsu Corporation, and TSF 4702, TSF 4703, TSF 4704, TSF 4705, TSF 4707, TSF 4708, TSF 4709, F42-B3115, SF 1708, SF 1923, SF 1921, SF 1925, OF TP AC3309, OF 7747, OF-NH TP AI3631,OF-NH TP AI3683 of GE Bayer Silicones, and organomodified silicones with amine and polyether functionality, commercially available under trade names such as XS69-B5476 of GE Bayer Silicones and Abilsoft AF100 of Goldschmidt.

Suitable polar functional substituents for inclusion in the functionalized silicones contain at least one class of oxygen-containing polar functional substituents, so that the oxygen content (% oxygen) in the summation of the one or more polar functional substituents (not containing the oxygen of the PDMS backbone) is 1% to 17%, preferably 2% to 15%, more preferably 3% to 13%, of the weight of the functionalized silicon. In addition, the hydrophilic functional silicone components of the present invention can have a silicone content (% silicone) of 45 to 95%, or 50 to 90%, or 55 to 85%, of the weight of the functionalized silicone. The silicone content, or the calculated percentage of silicone (% silicone), is defined as the average molecular weight of the PDMS backbone (consisting of silicon, oxygen, and all directly bound methyl groups) divided by the average molecular weight of the polymer as a whole. Similarly, the overall oxygen content (% oxygen) is defined as the molecular weight of each oxygen atom multiplied by the average number of oxygen atoms present on the silicone, subsequently divided by the average molecular weight of the polymer as a whole.

In addition, the functionalized silicone polymers can have polyoxyalkylene substituents. The polyoxyalkylene content (% polymer) can for example be from 5 to 55%, preferably from 10 to 50%, and more preferably from 15 to 45% of the weight of the polymer. Preferably, the sum of the percentage of silicone and the percentage of polyether is not 100%, so that amines and amides make up the remainder. The silicone content is defined above, and the polyether content (% polyether) is defined as the molecular weight of each polyether graft or block, multiplied by the average number of grafts or blocks, and divided by the average molecular weight of the polymer as a whole. If the graft or block polyether comprises both ethylene oxide (EO) and propylene oxide (PO) units, the % polyether indication includes the % EO and % PO. If the graft or block polyether comprises either only ethylene oxide (EO) units or only propylene oxide (PO) units, then the % polyether is equivalent to the % EO, or to the % PO.

A suitable functionalized silicone can for example correspond to the following formula:

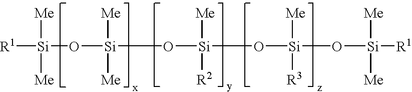

where Me stands for methyl: R¹ is methyl or R² or R³; R² is —(CH₂)ₐ—NH—[(CH₂)ₐ—NH]_{b}—H; and R³ is —(CH₂)ₐ—(OC₂H₄)ₘ—(OC₃H₆)ₙ—OZ; where x is approximately 50 to 1,500, y is approximately 1 to 20, z is approximately 1 to 20; a is approximately 2 to 5, preferably 2 to 4; b is 0 to 3, preferably 1; m is approximately 1 to 30; n is approximately 1 to 30, and Z is H, an alkyl group having 1-4 carbon atoms, or an acetyl group, with the reservation that if y is equal to 0, R¹ is an R² group, and if z is equal to 0, R^{1 is an R}³ group.

The graft-organomodified silicones, comprising amine and polyoxyalkylene groups having the formulas named above, can be manufactured using methods known to those skilled in the art, for example known polymerization reactions (e.g., equilibration or polycondensation) and known methods for depositing organic substituents on the silicone backbone (e.g. hydrosilylation).

In the following, exemplary structures of suitable functionalized silicones are described:

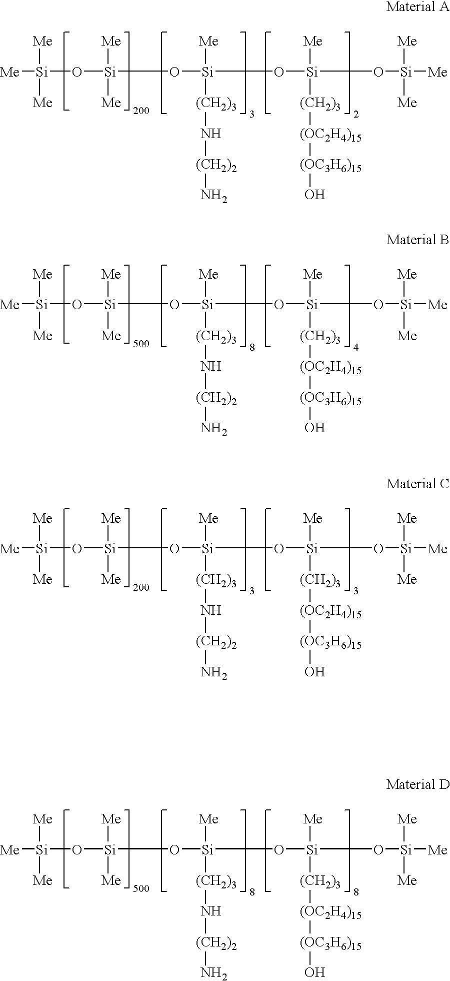

It is assumed that organosiloxane resins within the functionalized silicone fluid produce a three-dimensional network, resulting in viscoelasticity and improved adhesive characteristics on a fibrous substrate. For the case in which the composition for treating fibers is an emulsion, the mixture of the functionalized silicones and the organosiloxane resin can be dispersed therein in the form of emulsified droplets.

Organosiloxane resins, which can be contained in a composition corresponding to the present invention, comprise combinations of R₃SiO_{1/2} “M” units, R₂SiO “D” units, RSiO_{3/2} “T” units, SiO₂ “Q” units, for example in determined ratios to one another, so that for example the ratio RnSiO(4−n)/2 is fulfilled, where n is a value between 1.0 and 1.50 and R is a methyl group. Silanol or alkoxy functionalities can also be present.

For example, the organosiloxane resins comprise repeating monofunctional R₃SiO_{1/2} “M” units, and the quadrofunctional SiO₂ “Q” units, also known as “MQ” resins. In this case, the ratio of “M”- and “Q”-functional units is usefully 0.7 and the value of n is 1.2. Organosiloxane resins of this type are commercially obtainable as SR1000, available from GE Bayer Silicones and Wacker 803 of Wacker Silicones.

Usefully, the organosiloxane resins corresponding to the present invention are solid at 25° C. (77° F.) and have a molecular weight ranging from 1,000 g/mol (35.27 oz/mol) bis 10,000 g/mol (325.74 oz/mol).

Reference is made to Table 2, which demonstrates the improvement in hold that can be achieved by adding MQ resin to some commercially available functionalized silicones:

The following combinations can for example be used: silicone Z (an aminosilicone having an average of 110 D units and two terminal functional aminopropyl groups), silicone Z+1% MQ resin (obtainable as SR1000 from GE Silicones), silicone Z+5% MQ resin, silicone Z+10% MQ resin, XS69-B5476 (obtainable as XS69-B5476 from GE Silicones), XS69-B5476+0.5% MQ resin, XS69-B5476+1% MQ resin, XS69-B5476+2% MQ resin, XS69-B5476+10% MQ resin, DC-2-8566 (obtainable as DC-2-8566 from Dow Corning), DC-2-8566+0.05% MQ resin, Rhodorsil 21637 (obtainable as Rhodorsil 21637 from Rhodia), Rhodorsil 21637+0.5% MQ resin or Rhodorsil 21637+1.0% MQ resin, or mixtures of two or more of these.

The compound phenylpropyldimethylsilylsilicate, available for example under the trade name Silshine 151 from GE Bayer Silicones, has for example turned out to be particularly suitable in some cases.

The quantity of hydrophobic silicone oil in a composition according to the present invention is for example approximately 0.01 to approximately 10 wt %, or approximately 0.1 to approximately 8 wt %, or approximately 0.2 to approximately 3 wt %, in relation to the total weight of the composition in each case.

In the context of the present invention, hydrophobic silicone compounds having a refractive index of 1.0 or greater are particularly suitable. For example, hydrophobic silicone compounds having a refractive index of approximately 1.01 to approximately 1.6, in particular a refractive index of approximately 1.02 to approximately 1.55, or approximately 1.04 to approximately 1.5, or approximately 1.07 to approximately 1.45, or approximately 1.1 to approximately 1.41, or approximately 1.15 to approximately 1.38, or approximately 1.19 to approximately 1.34, or approximately 1.23 to approximately 1.30, or approximately 1.25 to approximately 1.29, are particularly suitable. The determination of the refractive index preferably takes place according to a standard method using a standard refractometer.

In the context of the present invention, the viscosity of suitable hydrophobic silicone compounds is in a range from approximately 10 to approximately 50,000 mPas. For example, hydrophobic silicone compounds having a viscosity from approximately 50 to approximately 40,000, or approximately 100 to approximately 35,000, or approximately 200 to approximately 25,000, or approximately 300 to approximately 15,000, or approximately 400 to approximately 10,000 mPas are particularly suitable. The determination of the viscosity takes place for example using a Haake VT550 viscosimeter with the measurement system Mv-Din at 25° C. (77° F.) and a shear rate of 50s⁻¹.

In addition to a hydrophobic silicone compound, a composition according to the present invention also contains at least one hydrophilic silicone compound.

In the context of the present invention, a “hydrophilic” silicone compound is understood to be a compound having a solubility in water that is at least as defined above.

The hydrophilic groups of the hydrophilic silicone compounds that are to be used according to the present invention are for example selected from hydroxyl groups, primary, secondary, or tertiary amino groups, quaternary ammonium groups, alkylene oxide groups, betainic groups, and thiosulfate groups.

For example, cation-active silicone compounds are suitable. These compounds are substituted with cationic groups or cationisable groups. Suitable cation-active silicone compounds have either at least one amino group or at least one ammonium group. Suitable silicone polymers having amino groups are known under the INCI designation Amodimethicone. These polymers are polydimethylsiloxanes with aminoalkyl groups. The aminoalkyl groups can be lateral or terminal.

Suitable aminosilicones are those having the general formula (I)

R¹R²R³Si-(OSiR⁴R⁵)ₓ-(OSiR⁶Q)_{y}-OSiR⁷R⁸R⁹ (I),

wherein R¹, R², R⁷ and R⁸ stand, independently of one another, for identical or different groups , for example 4 identical groups or 3 identical groups and one different group, or pairwise different groups, or two different groups and two different groups, or four different groups, and for C₁-C₁₀ alkyl or alkenyl or C₆-C₁₂ aryl or aralkyl, or for a hydroxyl group or for hydrogen, or for a C₁-C₁₀ alkoxy or acetoxy group, for example for C₁-C₄ alkyl, for example for methyl, R³ and R⁹ are, independent of one another, identical or different and stand for —(CR¹⁰₂)ₐ—NH₂, with a=1 to 6, C₁-C₁₀ alkyl, phenyl, hydroxy, hydrogen, C₁-C₁₀ alkoxy or acetoxy, preferably C₁-C₄ alkyl, particularly preferably methyl, R⁴, R⁵ and R⁶ are, independent of one another, the same or different, and stand for hydrogen, a linear or branched C₁-C₂₀ alkyl group that can contain O and N atoms, preferably C₁-C₁₀ alkyl or C₆-C₁₂ aryl or aralkyl, for example C₁-C₄ alkyl, in particular methyl, Q stands for -A-NR¹¹R¹² or -A-N⁺R¹¹R¹²R¹³, A standing for a divalent C₁-C₂₀ alkylene group that can also contain O and N atoms as well as hydroxyl groups, and R¹¹, R¹² and R¹³are, independently of each other, the same or different, and stand for hydrogen, a C₁-C₂₂ alkyl group, for example a C₁-C₄ alkyl group, or for C₆-C₁₂ aryl or aralkyl.

Q stands for example for —(CH₂)₃—NH₂, —(CH₂)₃NHCH₂CH₂NH₂, —(CH₂)₃OCH₂CHOHCH₂NH₂ or (CH₂)₃N(CH₂CH₂OH)₂, —(CH₂)₃ NH₃⁺ or —(CH₂)₃OCH₂CHOHCH₂N⁺(CH₃)₂R¹⁴, where R¹⁴ stands for a C₁-C₂₂ alkyl group that can also have hydroxyl groups, x stands for a number between 1 and 10,000, for example between 1 and 1,000; y stands for a number between 1 and 500, for example between 1 and 50.

The molecular weight of the aminosilicones is for example between 500 and 100,000. The amine portion (meq/g) is preferably in the range from 0.05 to 2.3, particularly preferably from 0.1 to 0.5.

Suitable silicone polymers having two terminal quaternary ammonium groups are known for example under the INCI designation Quaternium-80. These polymers are dimethylsiloxanes with two terminal aminoalkyl groups. Suitable quaternary aminosilicones are for example those having the general formula (II)

R¹⁵R¹⁶R¹⁷N+-A-SiR¹R²-(OSiR⁴R⁵)n-OSiR¹R²-A-N⁺R¹⁵R¹⁶R¹⁷ 2X⁻ (II)

A has the same meaning as indicated above in formula (I), and preferably stands for —(CH₂)₃OCH₂CHOHCH₂N⁺(CH₃)₂R¹⁴, where R¹⁴ has the meaning stated above, R¹, R², R⁴ and R⁵ have the same meaning as indicated above and stand for example for methyl, R¹⁵, R¹⁶, and R¹⁷ stand, independently of one another, for C₁-C₂₂ alkyl groups, which can contain hydroxyl groups, at least one of the groups having for example at least 10 C atoms, and the remaining groups having 1 to 4 C atoms; n stands for a number from 0 to 200, for example from 10 to 100.

Such diquatemary polydimethylsiloxanes are marketed for example by the company GOLDSCHMIDT (Germany) under the trade names Abil© Quat 3270, 3272 and 3274.

Suitable silicones having alkylene oxide groups are polydimethylsiloxanes having polyoxyalkylated substituents, in particular silicones modified with polypropylene oxide, polyethylene oxide, or a mixture thereof. The alkylene oxide groups can be lateral or terminal, or they can be polydimethylsiloxane/polyalkylene oxide block copolymers. The siloxanes modified with alkylene oxides are also designated as dimethylsiloxane glycol copolymers or as dimethicone copolyols. Suitable silicones having hydroxyl groups are hydrophilic dimethiconoles. These are hydrophilic polydimethylsiloxanes having hydroxyl end groups. Suitable silicones with thiosulfate groups are known under the INCI designation Dimethicone/Sodium PG-Propyldimethicone Thiosulfate Copolymer. The commercially available silicones having the designation SF1288, SF1188A (both from GE Bayer Silicone) or DC193 Surfactant (Dow Coming) are for example also suitable.

The quantity of hydrophilic silicone compound in a composition according to the present invention is for example approximately 0.01 to approximately 15 wt %, or approximately 0.1 to approximately 8 wt %, or approximately 0.5 to approximately 6 wt %, in relation to the total weight of the composition in each case.

In the context of the present invention, hydrophilic silicone compounds are particularly suitable that have, possibly in a suitable solution, a refractive index of 1.0 or greater. Particularly suitable are, for example, hydrophilic silicone compounds that have a refractive index of approximately 1.01 to approximately 1.8, in particular a refractive index of approximately 1.02 to approximately 1.75, or from approximately 1.05 to approximately 1.70, or from approximately 1.1 to approximately 1.68, or from approximately 1.15 to approximately 1.65, or from approximately 1.2 to approximately 1.62, or from approximately 1.23 to approximately 1.60, or from approximately 1.25 to approximately 1.58, or from approximately 1.28 to approximately 1.55, or from approximately 1.30 to approximately 1.52, or from approximately 1.33 to approximately 1.51, or from approximately 1.37 to approximately 1.50, or from approximately 1.40 to approximately 1.48, or from approximately 1.43 to approximately 1.45. The determination of the refractive index here takes place according to the methods already stated above. In some cases, hydrophilic silicone compounds have turned out to be particularly suitable that have a refractive index in a range from approximately 1.45 to approximately 1.57, for example a refractive index in a range from approximately 1.49 to approximately 1.53.

In the context of the present invention, the viscosity of suitable hydrophilic silicone compounds is in a range from approximately 10 to approximately 50,000 mPas. Particularly suitable are for example hydrophilic silicone compounds having a viscosity from approximately 50 to approximately 40,000, or approximately 100 to approximately 35,000, or approximately 200 to approximately 25,000, or approximately 300 to approximately 15,000, or approximately 400 to approximately 10,000 mPas. The determination of the viscosity here takes place for example according to one of the methods already named above.

In the context of the present invention, it has turned out to be advantageous if the hydrophobic silicone compound used, or a mixture that is used of two or more hydrophobic silicone compounds and the hydrophilic silicone compound that is used, or the mixture of two or more hydrophilic silicone compounds that are used, are selected such that the refractive indices of the two groups of silicone compounds are different. For example, in the context of the present invention groups of hydrophilic silicone compounds and hydrophilic silicone compounds are suitable if the refractive index of the hydrophilic silicone compounds is greater than the refractive index of the hydrophobic silicone compounds. In the context of a preferred specific embodiment of the present invention, the ratio of the refractive index of hydrophilic silicone compounds to hydrophobic silicone compounds is greater than 1; for example, a corresponding ratio of the refractive indices is between 1.0 and 2.0, for example between approximately 1.1 and approximately 1.9, or approximately 1.2 and approximately 1.8, or between approximately 1.3 and approximately 1.7, or between approximately 1.4 and approximately 1.6, for example approximately 1.5.

In addition to at least one hydrophobic silicone and at least one hydrophilic silicone, the composition according to the present invention also contains water. The quantity of water in a composition according to the present invention can be within broad limits. Suitable water contents are for example water contents from approximately 0.5 to approximately 99 wt %. As a rule, a composition according to the present invention has a water content from approximately 1 to approximately 80, for example approximately 2 to approximately 70 or approximately 5 to approximately 60 wt %; these indications relate to the weight of the composition as a whole.

In addition to the above-named compounds, a composition according to the present invention also contains a compound selected from the group of anionic surfactants, nonionic surfactants, cationic surfactants, polymers having cationic or cationisable groups, cationically derived proteins, cationically derived protein hydrolysates, and betaines.

If the compositions according to the present invention are intended for use for example as shampoos, then as a rule at least one anionic surfactant is present in addition to the combination of hydrophobic and hydrophilic silicones.

All anionic surface-active materials suitable for application to the human body are suitable for use together with the conserving combination of active ingredients according to the present invention. Such anionic, surface-active materials are characterized by an anionic group that confers solubility in water, such as for example at least one carboxylate, sulfate, sulfonate- or phosphate group, and at least one hydrocarbon group, preferably at least one lipophilic alkyl group having approximately 10 to approximately 22 C atoms. In addition, the molecule can contain glycol or polyglycolether groups, ester groups, ether groups, amide groups, and hydroxyl groups.

Examples of suitable anionic surfactants are linear fatty acids having 10 to 22 C atoms (soaps), -ethercarboxylic acids having the formula R¹⁸—O(—CH₂—CH₂O)ₓ—CH₂-COOH, in which R¹⁸ stands for a linear alkyl group having 10 to 22 C atoms and x stands for 0 or 1 to 16, amidethercarboxylates having the formula [R¹⁹—NH(—CH₂—CH₂—O)ₙ—CH₂—COO—]ₘZ, in which R¹⁹ stands for a linear or branched, saturated or unsaturated acyl group having 2 to 29 C atoms, n stands for whole numbers from 1 to 10, m stands for the numbers 1 or 2, and Z stands for a cation from the group of alkaline or alkali earth metals, acylsarcosides having 10 to 18 C atoms in the acyl group, acyl taurides having 10 to 18 C atoms in the acyl group, acyl isothionates having 10 to 18 C atoms in the acyl group, sulfosuccinic acid mono- and dialkylesters having 8 to 18 C atoms in the alkyl group and sulfosuccinic acid monoalkylpolyoxyethylesters having 8 to 18 C atoms in the alkyl group and 1 to 6 oxyethyl groups, linear alkanesulfonates having 12 to 18 C atoms, linear alpha-olefinsulfonates having 12 to 18 C atoms, alpha-sulfofatty acid methylesters of fatty acids having 12 to 18 C atoms, alkyl sulfates and alkylpolyglycolethersulfates having the formula R²⁰(—CH₂—CH₂O)ₓ—OSO₃H, in which R²⁰ stands for a linear or branched alkyl group having 10 to 18 C atoms and x stands for 0 or 1 to 12, sulfated hydroxyalkylpolyethylene- and/or hydroxyalkylpolypropylene glycolethers, sulfonates of unsaturated fatty acids having 12 to 24 C atoms and 1 to 6 double bonds, esters of tartaric acid and citric acid having alcohols that represent the additive products of approximately 2 to approximately 15 molecules of ethylene oxide and/or propylene oxide to fatty alcohols having 8 to 22 C atoms, each in the form of alkaline salts, alkaline earth salts, and/or ammonium salts, preferably in the form of their sodium salts, potassium salts, magnesium salts, and/or ammonium salts, as well as the mono-, di- and/or trialkanolammonium salts having 2 or 3 C atoms in the alkanol group.

Preferred anionic surfactants are alkyl sulfates, alkylpolyglycolethersulfates and ethercarboxylic acids having 10 to 18 C atoms in the alkyl group and up to 12 glycolether groups in the molecule, as well as sulfosuccinic acid mono- and -dialkylesters having 8 to 18 C atoms in the alkyl group and sulfosuccinic acid monoalkylpolyoxyethylesters having 8 to 18 C atoms in the alkyl group and 1 to 6 oxyethylene groups.

In the context of the present invention, in some cases it has proved useful to use ether sulfates, in particular lauryl ether sulfate, as anionic surfactants.

Besides the designated anionic surfactants, nonionic surfactants can also be present in the preparations according to the present invention.

As hydrophilic groups, these contain for example a polyol group, a polyalkylene glycolether group or a combination of polyol- and polyglycolether groups. Such compounds are for example additive products of 2 to 30 mole ethylene oxide and/or 0 to 5 mole propylene oxide to linear fatty alcohols having 8 to 22 C atoms or to fatty acids having 12 to 22 C atoms or to alkyl phenols having 8 to 15 C atoms in the alkyl group, C₁₂-C₂₂ fatty acid mono- and -diesters of additive products of 1 to 30 moles ethylene oxide to glycerin, C8-C₂₂alkylmono- and -oligoglycosides and their ethoxylated analogues, as well as additive products of 5 to 60 moles ethylene oxide to castor oil and/or hardened castor oil.

The compounds having used as surfactants and having alkyl groups can be pure substances. However, in manufacturing these materials, as a rule it is preferable to begin with native plant or animal raw materials, so that substance mixtures are obtained having a different number of hydrocarbon atoms in the alkyl chain, depending on the raw material used.

In the surfactants that represent additive products of ethylene and/or propylene oxide to fatty alcohols or derivates of these additive products, both products having a “normal” homologue distribution and also those having a narrowed homologue distribution can be used. Here, “normal” homologue distribution is understood as mixtures of homologues that are obtained in the conversion of fatty alcohol and alkylene oxide with the use of alkali metals, alkali metal hydroxides, or alkali metal alcoholates as catalysts. In contrast, restricted homologue distributions are obtained when for example hydrotalcites, alkaline earth metal salts of ether carboxylic acids , alkaline earth metal oxides, hydroxides, or alcoholates are used as catalysts. The use of products having restricted homologue distribution can be preferred.

In another specific embodiment, it is preferred to select as nonionic surfactants alkylpolyglycosides having the general formula R²⁰O(-Z)ₓ. In the compounds designated in this way, the alkyl group R²⁰ contains 6 to 22 carbon atoms, and can be both linear and branched. Preferred are primary linear alkyl groups, or alkyl groups that are methyl-branched at the 2-position. Such alkyl groups R²⁰ are for example 1-octyl-, 1-decyl-, 1-lauryl-, 1-myristyl-, 1-cetyl- and 1-stearyl groups. Especially preferred are 1-octyl-, 1-decyl-, 1-lauryl- or 1-myristyl groups. Given the use of what are known as “oxo-alcohols” as initial materials, compounds having an odd number of carbon atoms in the alkyl chain predominate.

The alkylpolyglycosides that can be used in the preparations according to the present invention can for example contain only one particular alkyl group R²⁰. Standardly, however, the alkylpolyglycosides are manufactured on the basis of natural fats and oils or mineral oils. In this case, mixtures corresponding to the initial compounds, or corresponding to the conversion of these compounds, are present as alkyl groups R²⁰.

As the sugar module Z, arbitrary mono- or oligosaccharides can be used. Standardly, sugars having 5 or 6 carbon atoms, as well as the corresponding oligosaccharides, are used. Such sugars are for example glucose, fructose, galactose, arabinose, ribose, xylose, lyxose, allose, altrose, mannose, gulose, idose, talose and sucrose. Preferred sugar modules are glucose, fructose, galactose, arabinose and sucrose; glucose is especially preferred.

In section, the alkylpolyglycosides that can be used according to the present invention contain 1.1 to 5 sugar units. Alkylpolyglycosides in which x stands for values from 1.1 to 1.6 are preferred. Very especially preferred are alkylpolyglycosides in which x is 1.1 to 1.4.

The alkoxylated homologues of the named alkylpolyglycosides can also be used according to the present invention. These homologues can contain on average up to 10 ethylene oxide and/or propylene oxide units per alkylglycoside unit.

In addition, a composition according to the present invention can contain a cation-active material, or two or more cation-active materials. The cation-active material is a substance that has an affinity for human hair on the basis of cationic or cationisable groups, in particular primary, secondary, tertiary, or quaternary amine groups. Suitable cation-active materials are selected from cationic surfactants, betainic surfactants, amphoteric surfactants, cation-active polymers having cationic or cationizable groups, cationically derived proteins, cationically derived protein hydrolysates, and betaine.

Suitable cation-active surfactants are surfactants that contain a quaternary ammonium group. These can be cationic or amphoteric, betainic surfactants. Cationic surfactants are especially preferred as a cation-active material. Suitable cationic surfactants contain amino groups or quaternary hydrophilic ammonium groups, which in solution carry a positive charge and can be represented by the general formula (III),

N⁺R²¹R²²R²³R²⁴X⁻ (III)

where R²¹ to R²⁴ stand, independently of one another, for aliphatic groups, aromatic groups, alkoxy groups, polyoxyalkylene groups, alkylamido groups, hydroxyalkyl groups, aryl groups or alkaryl groups having 1 to 22 C atoms, and X⁻ stands for an anion, for example a halogen, acetate, phosphate, nitrate, or alkyl sulfate, preferably a chloride. In addition to the carbon atoms and the hydrogen atoms, the aliphatic groups can also contain cross-compounds, or other groups, such as for example hydroxy groups or additional amino groups.

Examples of suitable cationic surfactants are the chlorides or bromides of alkyldimethylbenzyl ammonium salts, alkyltrimethyl ammonium salts, for example cetyltrimethyl ammonium chloride or -bromide, tetradecyltrimethyl ammonium chloride or -bromide, alkyldimethylhydroxyethyl ammonium chlorides or -bromides, the dialkyldimethyl ammonium chlorides or -bromides, alkylpyridinium salts, for example lauryl- or cetylpyridinium chloride, alkylamidoethyltrimethyl ammonium ether sulfates, and compounds having cationic character, such as aminoxides, for example alkylmethylamine oxides or alkylaminoethyldimethylamine oxides.

For example, cetyltrimethyl ammonium chloride is suitable, marketed for example in the form of a 26% aqueous solution under the trade name Dehyquart® A by the company Cognis AG, Düsseldorf (Germany), and under the trade name Genamin® CTAC by the company Clariant, Frankfurt (Germany), and in the form of a 50% solution in isopropanol under the trade name Arquad®16-50 by the company Akzo Chemicals GmbH, Düren (Germany).

Suitable amphoteric surfactants are derivates of aliphatic quaternary ammonium, phosphonium and sulfonium compounds having the formula (IV)

R²⁵-Y⁺(R²⁷)ₙ—CH₂—R²⁶-Z⁻ (IV)

where R²⁵ stands for a straight chain or branched chain alkyl, alkenyl, or hydroxyalkyl group having 8 to 18 carbon atoms and 0 to approximately 10 ethylene oxide units and 0 to 1 glycerin units; Y stands for a group containing N, P or S; R²⁷ stands for an alkyl or monohydroxyalkyl group having 1 to 3 carbon atoms; x stands for 1 if Y is a sulfur atom and x stands for 2 if Y stands for a nitrogen atom or a phosphorus atom; R²⁶ stands for an alkylene or hydroxyalkylene group having 1 to 4 carbon atoms, and Z⁻ stands for a carboxylate, sulfate, phosphonate or phosphate group.

Other amphoteric surfactants, such as betaines, are also suitable for the hair treatment agent according to the present invention. Examples of betaines include C8-C18 alkylbetaines, such as cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and lauryl-bis(2-hydroxypropyl)alphacarboxyethylbetaine; C8-C18-sulfobetaines such as cocodimethylsulfopropylbetaine, stearyldimethylsulfopropylbetaine, lauryldimethylsulfoethylbetaine, laurylbis-(2hydroxyethyl)sulfopropylbetaine; the carboxyl derivates of the imidazole, the C₈-C₁₈ alkyldimethyl ammonium acetates, the C₈- to C₁₈-alkyldimethylcarbonylmethyl ammonium salts, and the C₈-C18 fatty acid alkylamidobetaines, such as the coconut oil acid amidopropylbetaine, marketed for example in the form of a 30% aqueous solution under the trade name Tego® Betaine L7 by the company Goldschmidt AG, and the N-coconut oil acid amidoethyl-N-[2(carboxymethoxy)ethyl]-glycerin (CTFA name: Cocoamphocarboxyglycinate), which, for example, is sold by the Miranol Chemical Co. Inc. in the form of a 50% aqueous solution under the trade name Miranol® C2M Inc.

In the context of the present invention, it has turned out to be particularly suitable if a composition according to the present invention contains quaternary amidoalkyl compounds, for example coconut oil acid amidopropylbetaine.

In addition, in some cases it has turned out to be advantageous if a composition according to the present invention, if it contains for example anionic surfactants and quaternary amidoalkyl compounds, contains these two compound groups in a weight ratio of approximately 10:1 to approximately 1:1, in particular a weight ratio of approximately 8:1 to approximately 5:1.

The suitable cation-active polymers are preferably polymers that set or condition the hair. Suitable cation-active polymers preferably contain quaternary amine groups. The cation-active polymers can be homopolymers or copolymers, the quaternary nitrogen groups being contained either in the polymer chain or, preferably, as a substituent of one or more of the monomers. The ammonium group-containing monomers can be copolymerized with non-cationic monomers. Suitable cationic monomers are unsaturated, radically polymerizable compounds carrying at least one cationic group, in particular ammonium-substituted vinyl monomers such as for example trialkylmethacryloxyalkylammonium, trialkylacryloxyalkylammonium, dialkyldiallylammonium and quaternary vinyl ammonium monomers having groups containing cyclic cationic nitrogens, such as pyridinium, imidazolium or quaternary pyrrolidones, e.g. alkylvinylimidazolium, alkylvinylpyridinium, or alyklvinylpyrrolidone salts. The alkyl groups of these monomers are preferably lower alkyl groups, such as e.g. C1-C7 alkyl groups, especially preferably C1-C3 alkyl groups.

The ammonium group-containing monomers can be copolymerized with non-cationic monomers. Suitable comonomers are for example acrylamide, methacrylamide, alkyl- and dialkylacrylamide, alkyl- and dialkylmethacrylamide, alkylacrylate, alkylmethacrylate, vinylcaprolactone, vinylcaprolactam, vinylpyrrolidone, vinylester, e.g. vinylacetate, vinyl alcohol, propylene glycol or ethylene glycol, the alkyl groups of these monomers preferably being C₁-C₇ alkyl groups, particularly preferably C₁-C₃ alkyl groups.

Suitable polymers with quaternary amine groups are, for example, polymers described in the CTFA Cosmetic Ingredient Dictionary under the designations Polyquaternium such as methyl vinyl imidazolium chloride/vinyl pyrrolidone copolymer (Polyquatemium-16) or quaternized vinyl pyrrolidone/dimethylaminoethyl methacrylate copolymer (Polyquaternium-11),

Of the cationic polymers that can be contained in the agent according to the present invention the following, for example, are suitable: vinyl pyrrolidone/dimethylamino ethylmethacrylate methosulfate copolymer, sold under the trade names Gafquat® 755 N and Gafquat® 734 by Gaf Co. in the USA; Gafquat® 734 is especially preferred. Other cationic polymers include, for example, a copolymer sold by BASF in Germany under the trade name LUVIQUAT® HM 550 consisting of polyvinyl pyrrolidone and imidazolimine methochloride; a terpolymer sold by Calgon in the USA under the trade name Merquat® Plus 3300 consisting of dimethyl diallyl ammonium chloride, sodium acrylate, and acrylamide; a terpolymer from ISP in the USA sold under the trade name Gaffix® VC 713 consisting of vinyl pyrrolidone, dimethylamino ethyl methacrylate, and vinyl caprolactam; and the copolymer sold by Gaf under the trade name Gafquat® HS 100 consisting of vinyl pyrrolidone/methacrylamidopropyltrimethyl ammonium chloride.

Suitable cationic polymers that are derived from natural polymers are cationic derivatives of polysaccharides, for example, cationic derivatives of cellulose, starch, or guar. Furthermore, chitosan and chitosan derivatives are suitable. Cationic polysaccharides have the general formula (V)

G-O-B-N⁺R²⁸R²⁹R³⁰ X⁻ (V)

where G stands for an anhydroglucose group, for example starch- or cellulose anhydroglucose B for a divalent compound group, for example alkylene, oxyalkylene, polyoxyalkylene or hydroxyalkylene; R²⁸, R²⁹ and R³⁰ stand, independently of one another, for alkyl, aryl, alkylaryl, arylalkyl, alkoxyalkyl or alkoxyaryl each having 1 up to 18 C atoms, the total number of C atoms in R²⁸, R²⁹ and R³⁰ preferably being a maximum of 20; X⁻ stands for a standard counter-anion and has the same meaning as in formula (IV) and stands for example for chloride.

A cationic cellulose is sold by Amerchol under the name Polymer JR and has the INCI designation Polyquaternium-10. An additional cationic cellulose has the INCI designation Polyquaternium-24 and is sold by Amerchol under the trade name Polymer LM-200. A suitable cationic guar derivative is sold under the trade name Jaguar® R and has the INCI designation Guar Hydroxypropyltrimonium Chloride.

Particularly preferred cation-active materials are chitosan, chitosan salts and chitosan derivates. Chitosans that can be used in the present invention can be fully or partially deacetylated chitins. To produce a chitosan, one preferably starts with the chitin contained in the shell residues of crustaceans, which, as a cheaper and natural raw material, is available in large quantities. The molecular weight of the chitosans can be distributed over a broad spectrum, for example from 20,000 to approximately 5 million g/mole. For example, a low-molecular chitosan having a molecular weight of 30,000 g/mole (1,058.2 oz/mole) to 70,000 g/mole (2,469.2 oz/mole) is suitable.

Preferably, however, the molecular weight is greater than 100,000 g/mole (3,527.39 oz/mole), particularly preferably greater than 200,000 g/mole (7,054.79 oz/mole) to 700,000 g/mole (24,691.8 oz/mole). The level of deacetylation is preferably from 10 to 99%, with 60 to 99% being especially preferred.

A suitable chitosan is sold, for example, by Kyowa Oil&Fat in Japan under the trade name Flonac®. It has a molecular weight of 300,000 g/mole (10,582.2 oz/mole) to 700,000 g/mole (24,691.8 oz/mole), and is deacetylated to 70 to 80%. A preferred chitosan salt is chitosonium pyrrolidone carboxylate, which, for example, is sold under the name Kytamer PC by Amerchol in the USA. The contained chitosan has a molecular weight of approximately 200,000 g/mole (7,054.79 oz/mole) to 300,000 g/mole (10,582.2 oz/mole) and is deacetylated to 70 to 80%. Quaternated, alkylated, or hydroxyalkylated derivatives, for example, hydroxyethyl- or hydroxybutyl chitosan can be considered chitosan derivatives.

The chitosans or chitosan derivatives are preferably present in their neutralized or partially neutralized form. The level of neutralization for the chitosan or the chitosan derivative is preferably at least 50%, with 70 to 100% being especially preferred, based on the number of free base groups. As neutralization agents, in principle all cosmetically compatible inorganic or organic acids can be used, such as for example formic acid, tartaric acid, malic acid, lactic acid, citric acid, pyrrolidone carbide acid, hydrochloric acid, and others, of which pyrrolidone carbonic acid is particularly preferred.

Additional suitable cation-active, hair conditioning compounds include cationically modified protein derivates or cationically modified protein hydrolysates, and are for example known under the INCI designations Lauryldimonium Hydroxypropyl Hydrolyzed Wheat Protein, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein or Hydroxypropyltrimonium Hydrolyzed Wheat, Hydroxypropyltrimonium Hydrolyzed Casein, Hydroxypropyltrimonium Hydrolyzed Collagen, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyltrimonium Hydrolyzed Vegetable Protein.

Suitable cationically derived protein hydrolysates are substance mixtures, which, for example, can be obtained from glycidyl trialkyl ammonium salts or 3-halo-2-hydroxypropyl trialkyl ammonium salts via the conversion of alkaline, acidic, or enzyme hydrolyzed proteins. Proteins that are used as starting materials for the protein hydrolysates can be of plant or animal origin. Standard starting materials are, for example, keratin, collagen, elastin, soy protein, rice protein, milk protein, wheat protein, silk protein, or almond protein. The hydrolysis results in substance mixtures having mole masses in the range from approximately 100 to approximately 50,000. Standard average mole masses are in the range from approximately 500 to approximately 1000.

Advantageously, the cationically derived protein hydrolysates contain one or two long C8-C22 alkyl chains, and, correspondingly, two or one short C1-C4 alkyl chains. Compounds containing one long alkyl chain are preferred.

In addition, a composition according to the present invention can contain for example associative thickening agents.

A nonionic, amphipathic associative thickening agent is a polymer that contains both hydrophilic and also hydrophobic groups. Associative thickening agents are water-soluble polymers and have surfactant-type hydrophobic components that are able, in a hydrophilic (in particular aqueous) medium, to associate (i.e., enter into mutual interaction) both with themselves and also with other hydrophobic substances. The medium is thickened or gelled by the associative network that results from this.

Typically, associative thickening agents are manufactured through polymerization of polyethylene oxide pre-polymers and at least double-functional polycondensable substances such as e.g. isocyanates, mono- or diols having large aryl, alkyl or aryl/alkyl groups being built in in order to provide the hydrophobic modification. Preferred associative thickening agents are therefore hydrophobically modified polyalkylene glycols. Here, the hydrophilic component is preferably formed by polyoxyalkylene units, preferably polyoxyethylene units, but also polyoxypropylene units, or a mixture thereof. The hydrophobic component is preferably formed from hydrocarbon groups, e.g. long chain alkyl groups, alkylaryl or arylalkyl groups.

Suitable associative thickening agents are hydrophobically modified aminoplast-polyether copolymers. With regard to their structure and manufacture, reference is here made to WO 96/40815, whose disclosure with respect to associative thickening agents is to be regarded as part of the disclosure of the present application. In WO 96/40815, water-dispersible or water-soluble copolymers are described that are the reaction products of an acid-catalyzed polycondensation of at least doubly functional aminoplast monomers and at least doubly functional alkylene polyethers, as well as singly functional compounds having hydrophobic groups.

Suitable aminoplasts are shown in FIG. 1 of WO 96/40815. Especially preferred are the glycoluril derivates having formula X of WO 96/40815. Suitable alkylene polyethers are shown in FIG. 2 of WO 96/40815. Preferred alkylene polyethers are polyethylene oxide diols. These can have a level of ethoxylation of 20 to 500, preferably 50 to 350, especially preferably 100 to 250. Suitable singly functional compounds having hydrophobic groups are those having formula XIV in WO 96/40815.

The composition according to the present invention is preferably confectioned in an aqueous or aqueous-alcoholic milieu, and is distinguished in particular by its clarity and transparency. For this reason, the agent is advantageously also packaged in an optically corresponding packaging made of transparent or translucent material. Suitable packing materials include, in particular, glass and transparent or translucent plastics, such as e.g. polyethylene terephtalate.

As alcohols, in particular the lower alcohols standardly used for cosmetic purposes, having 1 to 4 carbon atoms, such as for example ethanol and isopropanol, can be contained. The water content is preferably from 40 to 95 wt %, especially preferably from 60 to 90 wt %. The alcohol content is preferably from 1 to 30 wt %, especially preferably from 5 to 20 wt %. Additional, especially preferred water-soluble solution or wet hold agents include polyvalent alcohols, in particular those having 2 to 4 carbon atoms, such as for example glycerin, ethylene glycol or propylene glycol in a quantity from 0.1 to 10 wt %, preferably from 0.5 to 5 wt %. Purely aqueous formulations are especially preferred.

In a preferred specific embodiment, the agent according to the present invention additionally contains at least one nonionic surfactant. Suitable nonionic surfactants are for example the nonionic emulsifiers stated in the “International Cosmetic Ingredient Dictionary and Handbook,” 7th ed., vol. 2, in the section “Surfactants—Emulsifying Agents.” Suitable nonionic surfactants are preferably selected from ethoxylated fatty acids having 10 to 26 carbon atoms, ethoxylated monovalent or polyvalent alcohols having 1 to 6 carbon atoms, ethoxylated fatty alcohols having 10 to 26 carbon atoms, ethoxylated hydrogenated or non-hydrogenated castor oil, alkylpolyglucosides, glyceride alkoxylates, fatty acid glycehdpolyalkylene glycolethers or fatty acid partial glyceride polyalkylene glycolethers having fewer than 30 alkylene glycol units, such as e.g. polyethylene glycol-(7)-glycerylcocoate, polyglycolamides, fatty acid sugar esters, ethoxylated fatty acid sugar esters and partial glycerides. The level of ethoxylation of ethoxylated surfactants is standardly from 1 to 400, preferably 2 to 200, especially preferably 3 to 25.

In a preferred specific embodiment, the agent according to the present invention contains only surfactants and emulsifiers that are water-soluble, i.e., surfactants that dissolve clear given a content of 1 wt % in water at 20° C. (68° F.).

Preferred nonionic surfactants are in particular fatty alcohol ethoxylates. For example, alcohols are suitable having 10 to 18, preferably 10 to 16, C atoms, and a level of ethoxylation of preferably 2 to 200, especially preferably from 3 to 25. The additional nonionic surfactants are preferably used in a quantity of 0.01 to 5 wt %.

In another specific embodiment, the agent according to the present invention can in addition contain at least one film-forming, hair-setting, synthetic or natural polymer. This additional polymer can have nonionic, anionic, or amphoteric character, and is used, if it is present, in a quantity from 0.5 to 10 wt %. Film-forming, hair-setting polymers are understood to be polymers that, when applied in 0.1 to 5% aqueous, alcoholic, or aqueous-alcoholic solution, are able to deposit a polymer film on the hair, and in this way to set the hair.

In addition, the agent according to the present invention can contain the additional components that are standard for hair treatment agents, e.g., non-setting nonionic polymers, non-setting anionic polymers, and non-setting natural polymers, as well as the combination thereof, preferably in a quantity of 0.01 to 10 wt %; perfume oils in a quantity of, preferably, 0.01 to 5 wt %; wetting agents or emulsifiers from the classes of the anionic, cationic, amphoteric or nonionogenic surface-active substances, in a quantity of, preferably, 0.01 to 10 wt %; humectants; preservatives, bactericidal and fungicidal active ingredients such as e.g. 2,4,4-Trichlor-2-hydroxydiphenylether, parabenes or methylchlorisothiazolinone, in a quantity of 0.01 to 1.0 wt %; buffering agents, such as e.g. sodium citrate or sodium phosphate, in a quantity from 0.1 to 1.0 wt %; coloring agents, such as for example fluorescein sodium salt, in a quantity from approximately 0.1 to 1.0 wt %; conditioners, such as e.g. plant and herb extracts, protein and silk hydrolysates, lanolin derivates, in a quantity of 0.1 to 5 wt %; sunblocking agents, antioxidants, radical scavengers, anti-dandruff active ingredients, fatty alcohols, shine agents, vitamins and moisturizers, in a quantity of 0.01 to 10 wt %.

Particularly suitable vitamins and vitamin derivatives are for example vitamin A, vitamin E, vitamin E-acetate, vitamin E-nicotinate, vitamin F, vitamin B₃, vitamin B₆, nicotinamide, vitamin H, vitamin C, vitamin B₅ and its derivatives, in particular panthenol, pantothenic acid, calcium pantothenate, pantothenyl ethyl ether, panthenyl hydroxypropyl steardimonium chloride (Panthequat®, Innovachem), pantethines and panthenyl triacetate. Of course, analogous derivatives can also be used, for example pantothenyl propyl ether, pantothenyl butyl ether, and other branched or linear, saturated or unsaturated homologues. The same holds for the salts of the pantothenic acids, whose possible counter-ions are not only limited to calcium, but can also include all physiologically acceptable metal cations, for example the alkali and alkali earth metals, in particular magnesium, sodium, or potassium. The present invention also includes the use of all possible stereoisomers of the various vitamins; in particular, for vitamin B₅ and its derivatives both the D- and the L-form, and all mixtures of the two forms, can be used according to the present invention.

Preferred is the use of vitamin C, vitamin H, vitamin E and its derivatives, as well as vitamin B₅ and its derivatives; the use of vitamin H, vitamin E-acetate, panthenol, Panthequat® and vitamin B₆ is especially preferred.

As a rule, the vitamins or vitamin derivatives are used in the preparations according to the present invention in quantities from 0.01 to 30 wt % in relation to the entire preparation. Standardly, for the manufacture of aqueous preparations ready for use, 0.02 to 15, in particular 0.02 to 8 wt %, is particularly advantageous. In many cases, quantities between 0.05 and 5 wt % are sufficient. For concentrates, it can be advantageous to use vitamins and/or vitamin derivatives in quantities from 0.05 to 30 wt %, in particular from 1 to 25 wt %, and especially preferably from 3 to 20 wt %.

The hair treatment agents according to the present invention are in particular shampoos, hair rinses, hair treatments, hair lotions or split-end repair fluids. Corresponding to the intended use, the preparations can be formulated as solutions, gels, creams, aerosols, or lotions. These can be both products that are rinsed out of the hair after a particular acting time, generally approximately 1 to 45 minutes, and products that are left on the hair.

The conserving combination of active ingredients according to the present invention can be used at different pH values. Thus, for example shampoos and hair rinses preferably have a pH value from 2.5 to 7.0, in particular from 4.0 to 6.0.

Practically any acid or base that can be used for cosmetic purposes can be used to set this pH value. For the case in which an acid is used to set the pH value, it can be preferred to use an acid from the group of edible acids, for example acetic acid, lactic acid, tartaric acid, citric acid, malic acid, ascorbic acid and gluconic acid.

Corresponding to the intended use and the type of formulation, the preparations according to the present invention can contain all cosmetic additives that are standard for the respective intended use.

Insofar as they have not been mentioned already in the present document, such standard additives are amphoteric surfactants, for example N-alkylglycines, N-alkylpropionic acids, N-alkylaminobutter acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids, each having approximately 8 to 18 C atoms in the alkyl group, zwitterionic surfactants, for example what are called the betaines, and 2-alkyl-3-carboxymethyl-3-hydroxyethyl-imidazoline, nonionic polymers, for example vinylpyrrolidone/vinylacrylate copolymers, polyvinylpyrrolidone and vinylpyrrolidone/vinylacetate copolymers, thickening agents such as agar-agar, guar gum, alginates, cellulose ethers, gelatins, pectins and/or xanthan gum, structurants such as glucose and maleic acid, hair-conditioning compounds such as phospholipides, for example soy lecithin, egg lecithin and kephalines, perfume oils, dimethylisosorbite and cyclodextrins, solubilizers such as ethanol, isopropanol, ethylene glycol, propylene glycol, glycerin and diethylene glycol, dyes, anti-dandruff ingredients such as climbazol, piroctone olamines and zinc omadines, additional substances for setting the pH value, active ingredients such as bisabolol, allantoin and plant extracts, sunblocking agents, greases and waxes such as spermaceti, beeswax, montan wax, paraffins, esters, glycerides and fatty alcohols, fatty acid alkanolamides, swelling and penetrating substances such as PCA, glycerin, propylene glycolmonoethylether, carbonates, hydrogen carbonates, guanidines, ureas, as well as primary, secondary and tertiary phosphates, opacifiers such as latex or styrene/acrylamide copolymers, nacreous lustre agents such as ethylene glycolmono- and -distearate or PEG-3-distearate, propellants such as propane-butane mixtures, N₂O, dimethylether, CO₂ and air, as well as antioxidants.

The agent according to the present invention can be present for example in a pH range from 2.0 to 9.5. Weakly acidic pH values, in the range between 4.5 and less than 7, or from 5.5 to 6.5, are for example suitable.

If the composition according to the present invention is used as a hair treatment, it can be provided as a lotion, a thickened lotion, a liquid gel, or as a high-viscosity gel. Preferably, in this case it is present in a medium-viscosity form; i.e., it preferably has the consistency of a thickened lotion or a liquid gel. If it is provided in a low-viscosity form, it can also be sprayed onto the hair in order to achieve a particularly good distribution. The hair treatment agent according to the present invention is then provided in combination with a suitable mechanically operated spraying device. Mechanical spraying devices are to be understood as devices that enable the spraying of a liquid without using a propellant. As a suitable mechanical spray device, for example a spray pump, or a container provided with an elastic spray valve and in which the cosmetic agent according to the present invention is filled under pressure, can be used, the elastic container expanding, and the agent being continuously dispensed when the spray valve is open due to the contraction of the elastic container.

The composition according to the present invention is for example used as a hair treatment in that a quantity sufficient for the desired conditioning effect is distributed in or on the dry hair, or is distributed in or on the wet or moist hair after the hair has been washed. The quantity to be applied depends on the fullness of the hair, and is typically 1 g (0.04 oz) to 25 g (0.9 oz). In the case of use as a rinse product, the hair is rinsed after a sufficient acting time, for example 1 to 15 minutes. Subsequently, the hair is combed through if necessary, or is styled and dried. In the case of use as a leave-in product, the hair is not rinsed after the application of the agent.

In specific embodiments, a composition according to the present invention can for example have the following compositions:

Hair rinse:

The manufacture of the compositions according to the present invention takes place in the context of the standard procedure. In principle, in the present case all types of manufacture may be used, as long as they result, after mixing of the corresponding compounds, in the composition having the desired product characteristics.

In the following, the present invention is explained in more detail on the basis of sample formulas.

Sample formulas:

Example 1: Hair rinse

| | | |
|---|---|---|
| 4.3 g | (0.2 oz) | cetearyl alcohol (Lanette ® O) |
| 0.4 g | (0.01 oz) | cetyl lactate |
| 0.5 g | (0.02 oz) | Vaseline |
| 1.2 g | (0.04 oz) | cetyltrimethyl ammonium chloride |
| 0.45 g | (0.02 oz) | polyvinylpyrrolidone |
| 1 g | (0.04 oz) | hydrophilic silicone (e.g. Mirasil aDM-E |
| | | (Rhodia), abilsoft AF 100 (Goldschmidt)) |
| 0.5 g | (0.02 oz) | hydrophobic silicone (e.g. Dow Corning 200, |
| | | Silshine 151) |
| balance to 100 g | (3.53 oz) | water |

Example 2: Hair conditioner

| | | |
|---|---|---|
| 5.5 g | (0.2 oz) | cetearyl alcohol (Lanette ® O) |
| 1.2 g | (0.04 oz) | Vaseline |
| 1.0 g | (0.04 oz) | Paraffinum Liquidum |
| 0.5 g | (0.02 oz) | dimethylpolysiloxane (Belsil ® DM 500) |
| 0.3 g | (0.01 oz) | lanolin alcohol |
| 0.2 g | (0.007 oz) | lanolin |
| 1.2 g | (0.04 oz) | cetyltrimethyl ammonium chloride |
| 1 g | (0.04 oz) | hydrophilic silicone (e.g. Mirasil ADM-E (Rho- |
| | | dia), abilsoft AF 100 (Goldschmidt)) |
| 0.5 g | (0.02 oz) | hydrophobic silicone (e.g. Dow Corning 200, |
| | | Silshine 151) |
| 0.3 g | (0.01 oz) | citric acid |
| 0.4 g | (0.01 oz) | perfume |
| balance to 100 g | (3.53 oz) | water |

Example 3: Creme-type hair treatment

| | | |
|---|---|---|
| 6 g | (0.2 oz) | cetearyl alcohol |
| 1.7 g | (0.06 oz) | glycerin |
| 1 g | (0.04 oz) | cetyltrimethyl ammonium chloride |
| 1 g | (0.04 oz) | vegetable oil |
| 0.5 g | (0.02 oz) | panthenol |
| 1 g | (0.04 oz) | hydrophilic silicone (e.g. Mirasil ADM-E |
| | | (Rhodia), abilsoft AF 100 (Goldschmidt)) |
| 0.5 g | (0.02 oz) | hydrophobic silicone (e.g. Dow Corning 200, |
| | | Silshine 151) |
| 0.2 g | (0.007 oz) | perfume |
| balance to 100 g | (3.53 oz) | Water |

Example 4: Leave-on spray hair treatment

| | | |
|---|---|---|
| 0.5 g | (0.02 oz) | cetyl alcohol |
| 0.3 g | (0.01 oz) | glycerin |
| 0.25 g | (0.009 oz) | cetyltrimethyl ammonium chloride |
| 0.2 g | (0.007 oz) | styrene/vinylpyrrolidone copolymer |
| 0.2 g | (0.007 oz) | panthenol |
| 1 g | (0.04 oz) | hydrophilic silicone (e.g. Mirasil ADM-E |
| | | (Rhodia), abilsoft AF 100 (Goldschmidt)) |
| 0.5 g | (0.02 oz) | hydrophobic silicone (e.g. Dow Corning 200, |
| | | Silshine 151) |
| 0.2 g | (0.007 oz) | perfume |
| 4.8 g | (0.2 oz) | ethanol |
| balance to 100 g | (3.53 oz) | Water |

What is claimed is:

1. Composition, containing
a) 0.01 to 10 wt % of a hydrophobic silicone compound, and
b) 0.01 to 15 wt % of at least one hydrophilic silicone compound, and
c) water, as well as at least one compound selected from the group consisting of anionic surfactants, nonionic surfactants, cationic surfactants, polymers having cationic or cationizable groups, cationically derived proteins, cationically derived protein hydrolysates, and betaines.

2. Composition according to claim 1, characterized in that the hydrophilic silicone compound is selected from the group consisting of silicone compounds having cationic groups, hydroxy-substituted siloxanes, siloxane/polyoxyalkylene copolymers, and amino-substituted siloxanes.

3. Composition according to claim 1 or 2, characterized in that it contains as a hydrophobic silicone compound a compound from the group of the organosilicates, having the general formula ((R)₂SiO{(R)₂SiO }_{y}Si(R)₃, where R stands for a linear or branched, saturated or unsaturated alkyl group having 1 to 6 C atoms or for a mono- or polycyclic cycloalkyl- or heterocycloalkyl group having 4 to 8 C atoms, or for an aromatic or araliphatic group having 6 to 10 C atoms, and y stands for a number from 0 to 5.

4. Composition according to one of claims 1 to 3, characterized in that the hydrophilic silicone compound has a functional group that is selected from the group consisting of hydroxyl groups, primary, secondary, or tertiary amino groups, quaternary ammonium groups, alkylene oxide groups, betainic groups and thiosulfate groups.

5. Composition according to one of claims 1 to 4, characterized in that it is present in an optically clear or at least translucent form.

6. Composition according to one of claims 1 to 5, characterized in that the refractive index of the hydrophilic silicone compound is greater than the refractive index of the hydrophobic silicone compound.

7. Composition according to one of claims 1 to 6, characterized in that the viscosity of the hydrophobic silicone compound is 10 to 50,000 mPas.

8. Composition according to one of claims 1 to 7, characterized in that it is present in a transparent or translucent packaging.

9. Use of a mixture containing
a) 0.01 to 10 wt % of a hydrophobic silicone compound, and
b) 0.01 to 15 wt % of at least one hydrophilic silicone compound, and
c) water, as well as at least one compound selected from the group consisting of anionic surfactants, cationic surfactants, polymers having cationic or cationisable groups, cationically derived proteins, cationically derived protein hydrolysates, and betaine, for the manufacture of a cosmetic agent.

10. Method for manufacturing a composition according to one of claims 1 to 7, characterized in that
a) 0.01 to 10 wt % of a hydrophilic silicone compound, and
b) 0.01 to 15 wt % of at least one hydrophilic silicone compound, and
c) water, as well as at least one compound selected from the group consisting of anionic surfactants, cationic surfactants, polymers having cationic or cationisable groups, cationically derived proteins, cationically derived protein hydrolysates, and betaine, are mixed.
